(19) United States
(12) Patent Application Publication
(10) Pub. No.: US 2025/0134020 A1
(43) Pub. Date: May 1, 2025

(54) Systems And Methods For Processing Explant Material

(71) Applicant: MONSANTO TECHNOLOGY LLC, Saint Louis, MO (US)

(72) Inventors: Saad ALHAJERI, Saint Louis, MO (US); Richard BAILEY, High Ridge, MO (US); Joshua Matthew KARY, Wentzville, MO (US); Andrew SNYDER, Wentzville, MO (US); Emanda TRAMBLE, Saint Louis, MO (US)

(21) Appl. No.: 18/932,562

(22) Filed: Oct. 30, 2024

Related U.S. Application Data
(60) Provisional application No. 63/546,868, filed on Nov. 1, 2023.

(51) Int. Cl. A01H 4/00

(52) U.S. Cl. CPC A01H 4/001

(57) ABSTRACT

Automated systems and methods are provided for use in processing explant material. One example system includes a first station configured to sterilize explant material with sterilization media, a second station configured to receive the explant material from the first station and rinse the explant material, and a third station configured to receive the explant material from the second station and hydrate the explant material with rehydration media. The system also includes a support structure, wherein the first station, the second station, and the third station are positioned on the support structure.

### CROSS-REFERENCE TO RELATED APPLICATION

This application claims the benefit of, and priority to, U.S. Provisional Application No. 63/546,868, filed on Nov. 1, 2023. The entire disclosure of the above-referenced application is incorporated herein by reference.

### FIELD

The present disclosure generally relates to systems and methods for processing explant material.

### BACKGROUND

This section provides background information related to the present disclosure which is not necessarily prior art.

Development of transgenic crops often requires plant explant materials capable of being genetically transformed and regenerated into a transgenic plant, which is then capable of passing a transgene to progeny. For instance, in corn, individual embryos may be removed to provide the explant material for use in such transformation.

### SUMMARY

This section provides a general summary of the disclosure, and is not a comprehensive disclosure of its full scope or all of its features.

Example embodiments of the present disclosure generally relate to automated systems for use in processing explant material. In one example embodiment, such a system generally includes a first station configured to sterilize explant material with sterilization media; a second station configured to receive the explant material from the first station and rinse the explant material; a third station configured to receive the explant material from the second station and hydrate the explant material with rehydration media; and a support structure, wherein the first station, the second station, and the third station are positioned on the support structure.

Example embodiments of the present disclosure also generally relate to automated methods for use in processing explant material. In one example embodiment, such a method generally includes sterilizing explant material within a reservoir of a first station of an automated explant processing system using a sterilization media; actuating, by a computing device, the reservoir to transfer the sterilized explant material from the reservoir to a float tank of the system; rinsing the explant material received within the float tank; directing, via fluid flow within the float tank, the explant material from the float tank to a rehydration tank; and hydrating the explant material received in the rehydration tank with a rehydration media.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of an automated system for use in processing explants and including one or more aspects of the present disclosure;

FIG. 2 is another perspective view of the system of FIG. 1;

FIG. 3 is a rear elevation view of the system of FIG. 1;

FIG. 4 is a side elevation view of the system of FIG. 1;

FIG. 5 is a top plant view of the system of FIG. 1;

FIGS. 6 and 7 are front elevation views of the system of FIG. 1;

FIG. 8 is a fragmentary front elevation view of the system of FIG. 1, illustrating a sterilization station, a rinse and floatation station, and a rehydration station of the system;

FIGS. 9 and 10 are perspective views of the sterilization station of the system of FIG. 1;

FIG. 11 is a perspective view of the rinse and floatation station of the system of FIG. 1;

FIGS. 12 and 13 are perspective views of a float tank of the rinse and floatation station of FIG. 11;

FIGS. 14 and 15 are perspective views the rehydration station of the system of FIG. 1;

FIG. 16 is a perspective view of a rehydration tank of the rehydration station of FIG. 14;

FIGS. 17 and 18 are perspective views of the system of FIG. 1, with a module of the system shown removed from a support structure of the system;

FIGS. 19-24 are example charts illustrating comparisons of explant material processed by the automated system of FIG. 1 and processed by conventional manual operations; and

FIG. 25 is an example computing device that may be used in the automated system of FIG. 1.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

Explant material (or explants) provides plant tissue for use in transformation and regeneration of modified plants. In connection therewith, the explant material generally includes transformable and regenerable tissue (e.g., embryonic and/or meristematic tissue, etc.), for example, capable of being genetically transformed and regenerated into a transgenic plant. To obtain such explant material, seeds from desired plants are initially collected and sanitized. The sanitized seeds are then dried to a desired moisture content and explant material is excised or removed from the seeds. In turn, the explant material may be enriched and purified by removing debris and unnecessary seed parts, and then further dried to obtain a desired moisture content for storage.

In connection with the above, the systems and methods herein provide for preparation of explant material, after the explant material is removed from the seeds, for subsequent use (e.g., transformation and culturing, etc.). This preparation generally includes performing one or more of a sterilization operation, a rinsing operation, a floatation operation, and a rehydration operation on the explant material. Uniquely, the systems and methods herein provide for automation of such operations, for example, to improve consistency (and/or reduce variability) in quality of the resulting explant material, while also maintaining viability for the subsequent use.

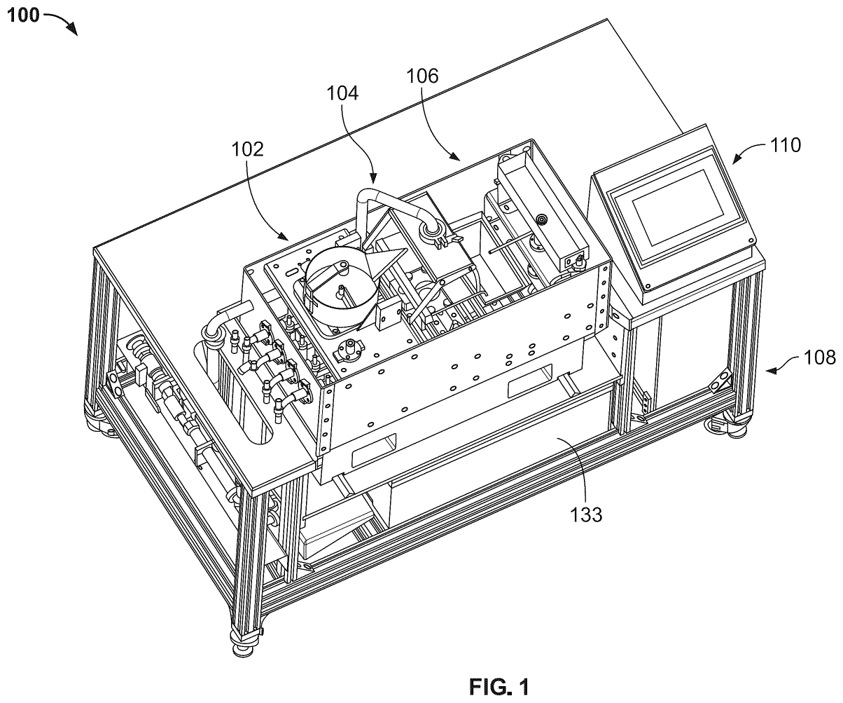

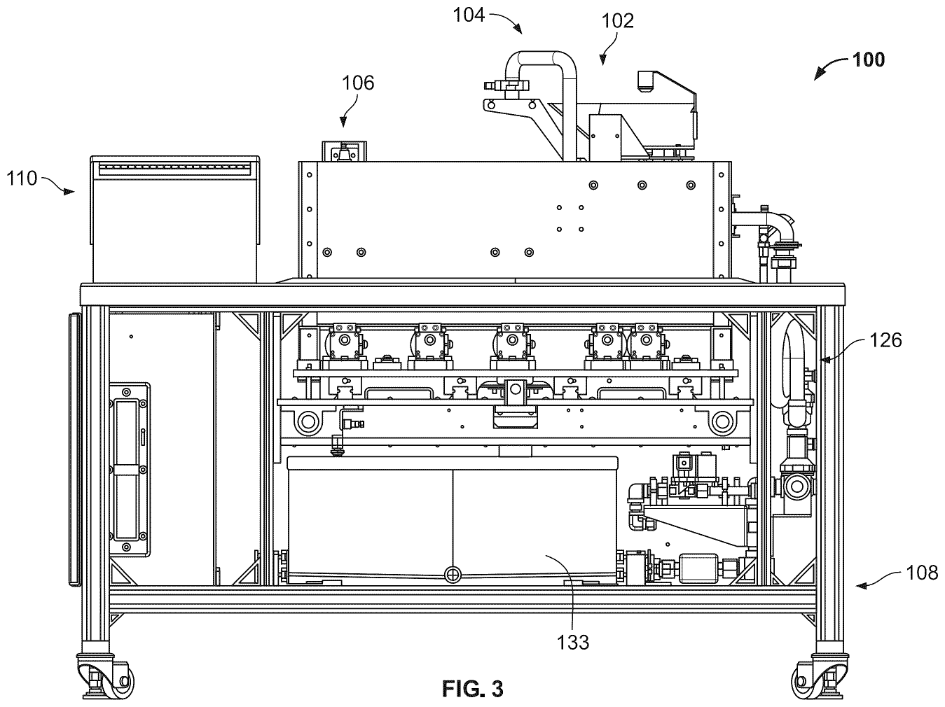

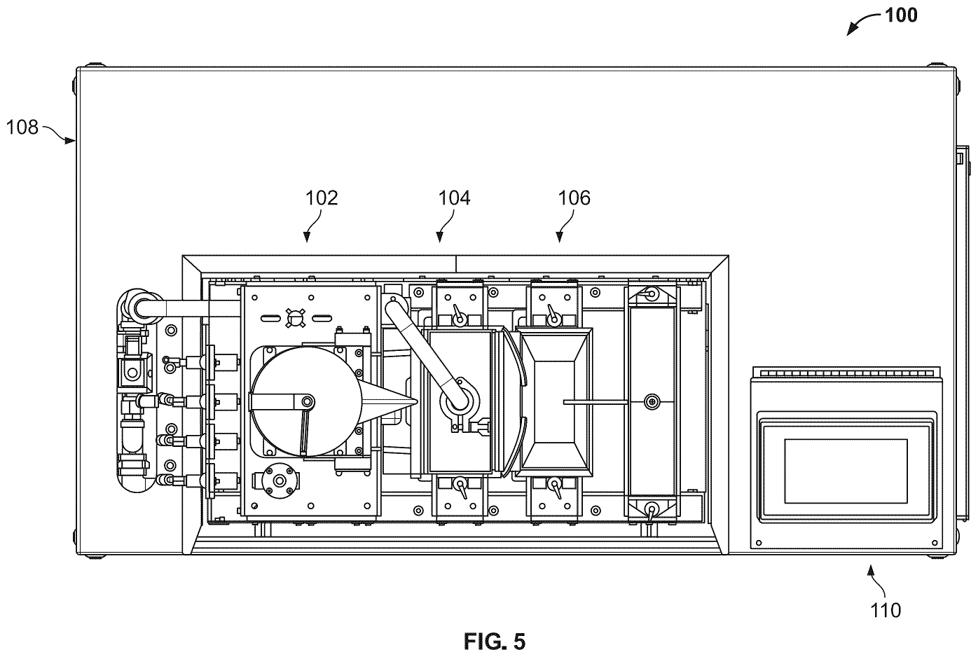

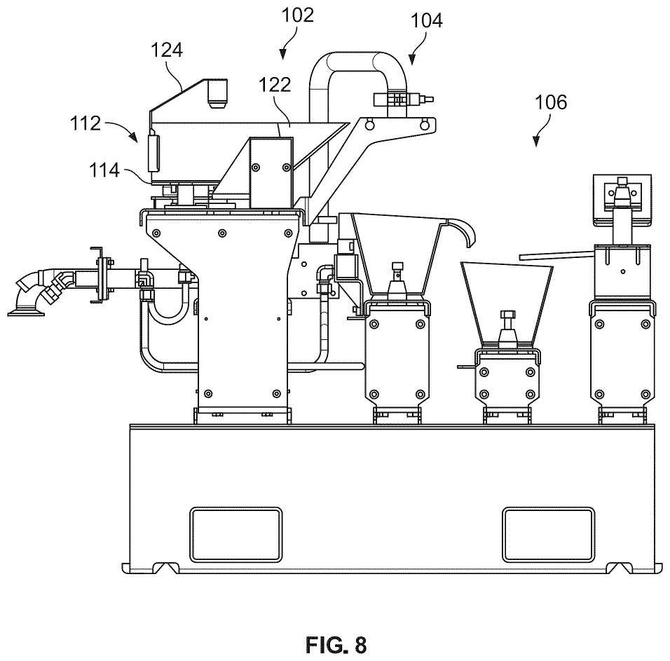

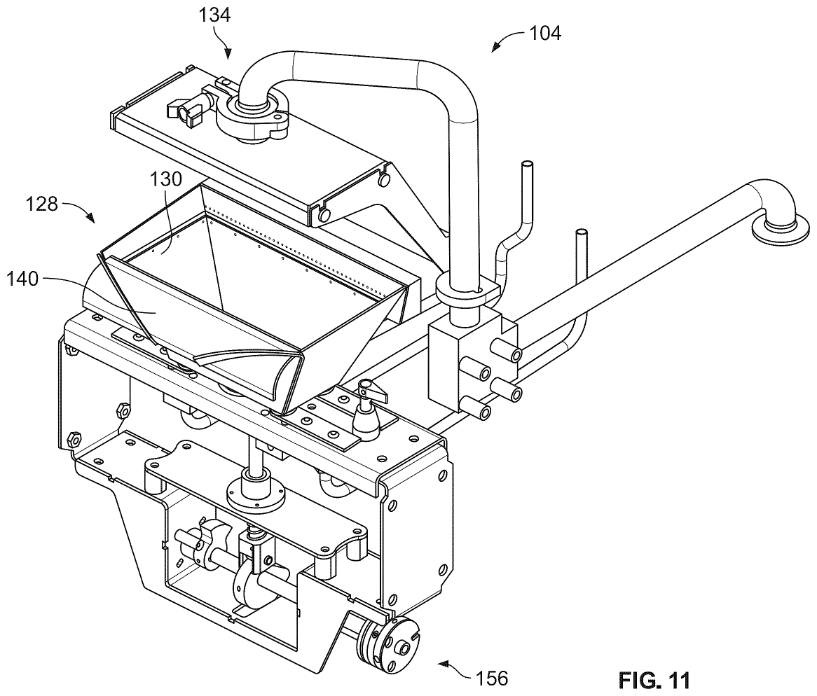

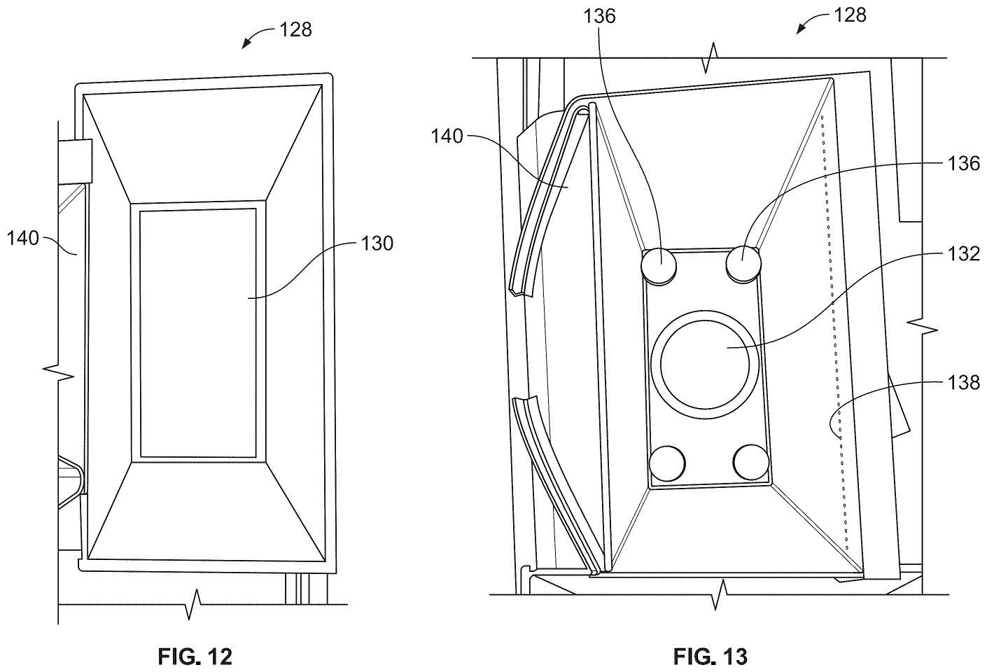

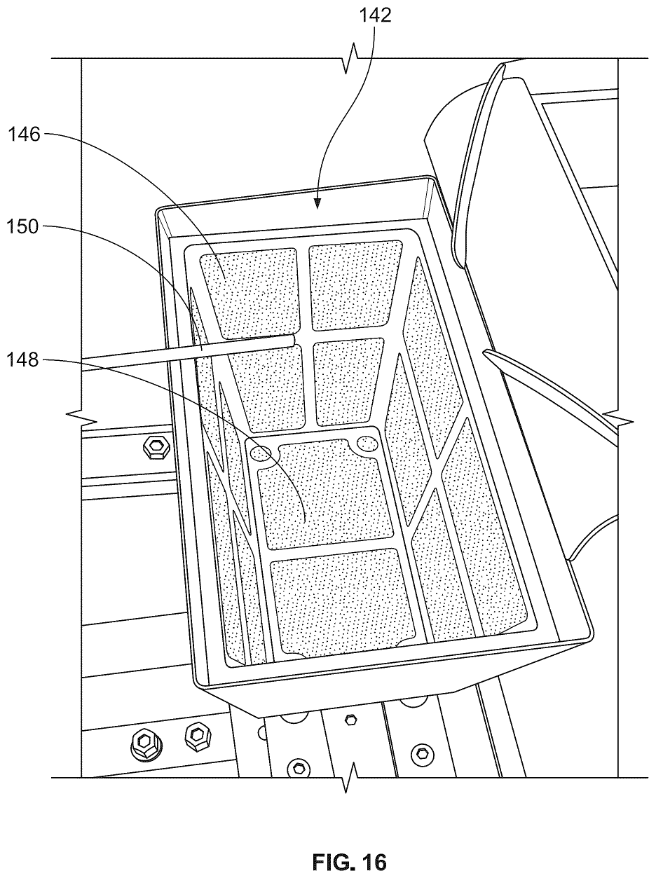

FIGS. 1-18 illustrate an example embodiment of an automated explant preparation system 100 including one or more aspects of the present disclosure. The system 100 is configured to prepare explant material for subsequent use in transformation and culturing operations. The explant material introduced into the system 100 may be obtained directly from seeds and then introduced to the system 100 (e.g., without being subsequently stored, etc.), or it may include explant material obtained from storage (e.g., explant material previously removed from seeds and then stored prior to being introduced to the system 100, etc.).

As shown in FIGS. 1-5, the system 100 generally includes a sterilization station 102 (e.g., a first station), a rinse and floatation station 104 (e.g., a second station), and a rehydration (or hydration) station 106 (e.g., a third station) each positioned within, or mounted on, a support structure 108 (e.g., a cart, a table, etc.). In connection therewith, the sterilization station 102 is configured to sterilize the explant material, for example, so that surfaces of the explant materials are free of microorganisms or other material/debris that may affect culturing, etc. The rinse and floatation station 104 is configured to rinse the sterilized explant material, to remove any remaining sterilization media therefrom, and to help separate any non-explant material (e.g., debris, other seed parts, etc.) from the explant material (e.g., via floating the explant material, etc.). And, the rehydration station 106 is configured to hydrate (or rehydrate) the explant material, for example, to a desired moisture content (or moisture content range) in preparation for transformation and culturing, etc.

The system 100 also includes a computing device 110 configured to control one or more operations of the sterilization station 102, the rinse and floatation station 104, and the rehydration station 106 (and to facilitate automation thereof). The computing device 110 is also configured to control interactions between the stations 102, 104, 106 to facilitate automated movement of the explant material therebetween. In the illustrated embodiment, the computing device 110 is illustrated as coupled to (e.g., positioned on, etc.) the support structure 108. In other embodiments, though, the computing device 110 may be remote from (or apart from) the support structure 108, but still configured to control one or more operations of the sterilization station 102, the rinse and floatation station 104, and the rehydration station 106 (and to still facilitate automation thereof). In connection therewith, the computing device 110 may be configured to operate one or more preprogrammed cycles, one or more customizable cycles, etc., whereby various parameters of the system 100 may be altered, etc.

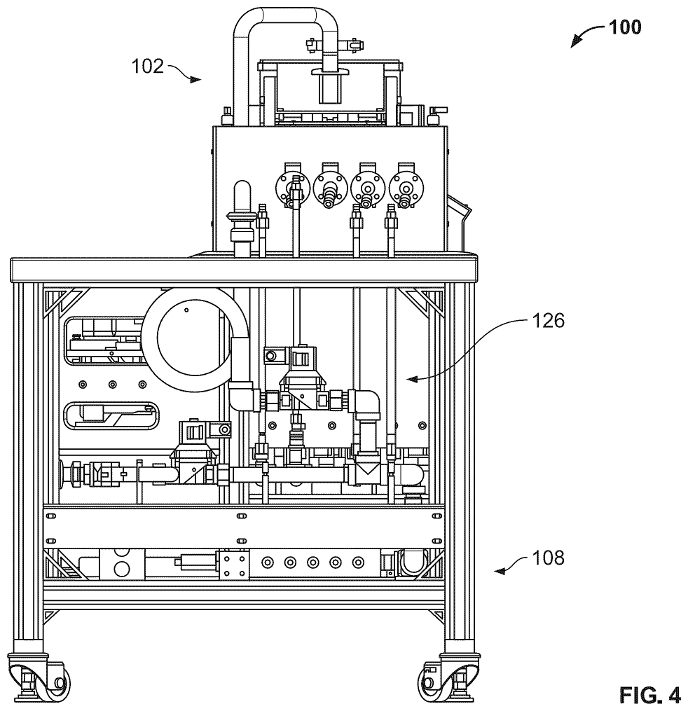

In the illustrated embodiment, the rinse and floatation station 104 is disposed generally between the sterilization station 102 and the rehydration station 106. As such, explant material may be initially introduced into the sterilization station 102 and then may sequentially flow, or be transferred or transported, in an automated manner (e.g., via instruction by the computing device 110, etc.), from the sterilization station 102 to the rinse and floatation station 104 and then to the rehydration station 106. In addition, as further shown in FIG. 4, the sterilization station 102 is located generally above (e.g., generally higher than, etc.) the rinse and floatation station 104, and the rinse and floatation station 104 is located generally above (e.g., generally higher than, etc.) the rehydration station 106. As such, in the illustrated embodiment, explant material introduced to the sterilization station 102 (after sterilization is complete) may subsequently be transferred (e.g., poured, etc.) from the sterilization station 102 to the rinse and floatation station 104 via use of gravity and/or flowing water (e.g., gravity alone, flowing water alone (e.g., flowing sterile water, etc.), or gravity and flowing water in combination, etc.). Similarly, after being received at the rinse and floatation station 104, the explant material may subsequently be transferred (e.g., floated, directed, flowed, etc.) to the rehydration station 106 via gravity. These operations will be described in more detail hereinafter.

In some example embodiments, heat may be applied during the rehydration operation (e.g., as a heat treatment, etc.), via a heating element which is functionally connected to the rehydration station 106. The conditions of the heat treatment may appropriately be selected, for example, depending on a type of the plant used (for the explant material) and the like, and may usually be carried out at a temperature between about 30° C. and about 60° C., preferably between about 33° C. and about 55° C., and more preferably between about 37° C. and about 52° C. In addition, the time of the heat treatment may appropriately be selected, for example, depending on the heating temperature, type of the plant used, type of the cells or tissue to be heat-treated and so on, and may be between about 5 seconds and about 24 hours. When the heating temperature is relatively high (e.g., based on the above ranges, etc.), the efficiency of transferring genes may be significantly promoted even if the time of the heat treatment is relatively short (e.g., based on the above ranges, etc.). For instance, when the heating temperature is about 60° C., heat treatment for about 5 seconds may significantly promote the efficiency of gene transfer. On the other hand, when the heating temperature is as low as about 34° C., the efficiency of gene transfer may be promoted by heat treatment for several tens of hours. In most cases, particularly preferred heating conditions may be between about 37° C. and about 52° C. for between about 1 minute and about 24 hours. The appropriate heating conditions for the particular plant cells or tissue may be selected by a routine experiment, etc. That said, by heating the plant cells or plant tissue at a temperature not lower than about 55° C. for a relatively longer time, the plant cells may be damaged and the efficiency of transformation may be decreased. Therefore, when the heating temperature is not lower than about 55° C., the heating time is preferably relatively shorter, for example, not longer than about 3 minutes, and preferably not longer than about 1 minute, so as to avoid damaging of the plant cells.

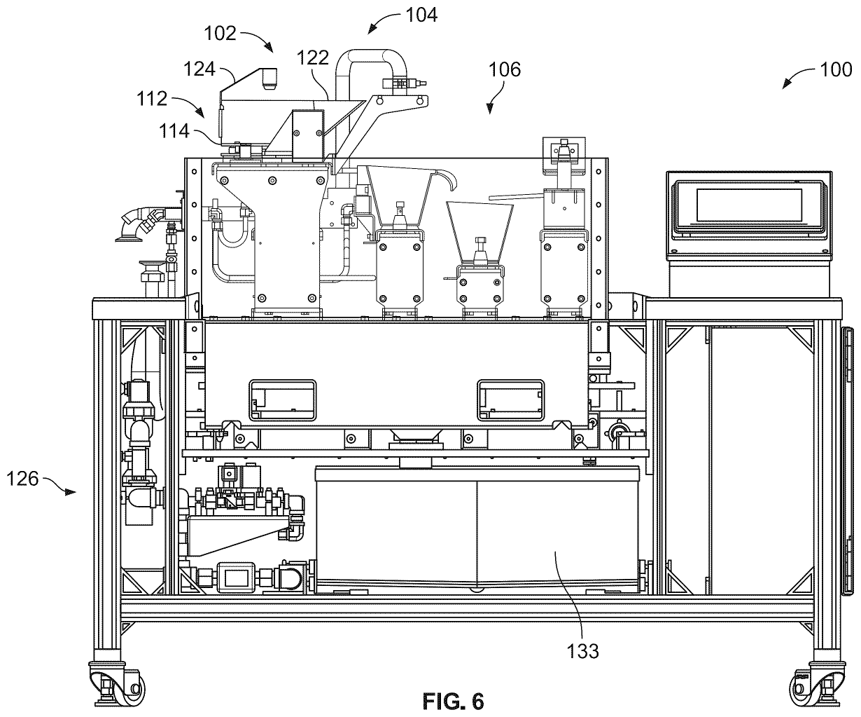

With reference now to FIGS. 6-10, the sterilization station 102 includes a sterilization reservoir 112 (or container) mounted on (or coupled to) a tray 114 (broadly, a support). The sterilization reservoir 112 is configured to hold, retain, etc. explant material therein in preparation for the automated processing described herein. A paddle 116 is disposed within the sterilization reservoir 112 and is configured to rotate (e.g., oscillate, rotate, move, etc.) within the reservoir 112 to mix (or stir) the explant material with sterilization media (also delivered to the sterilization reservoir 112 with the explant material, or before or after).

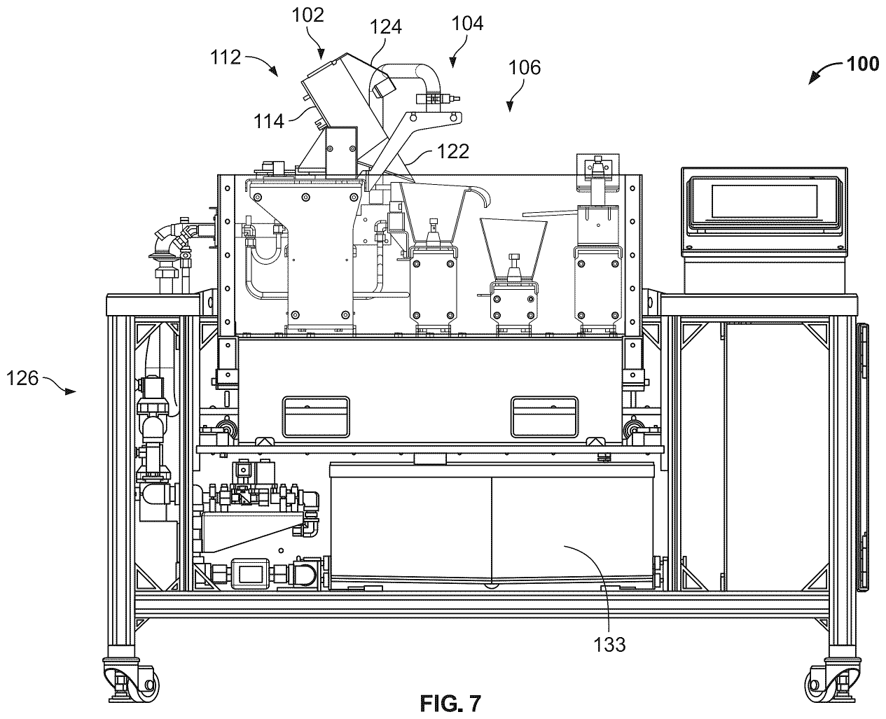

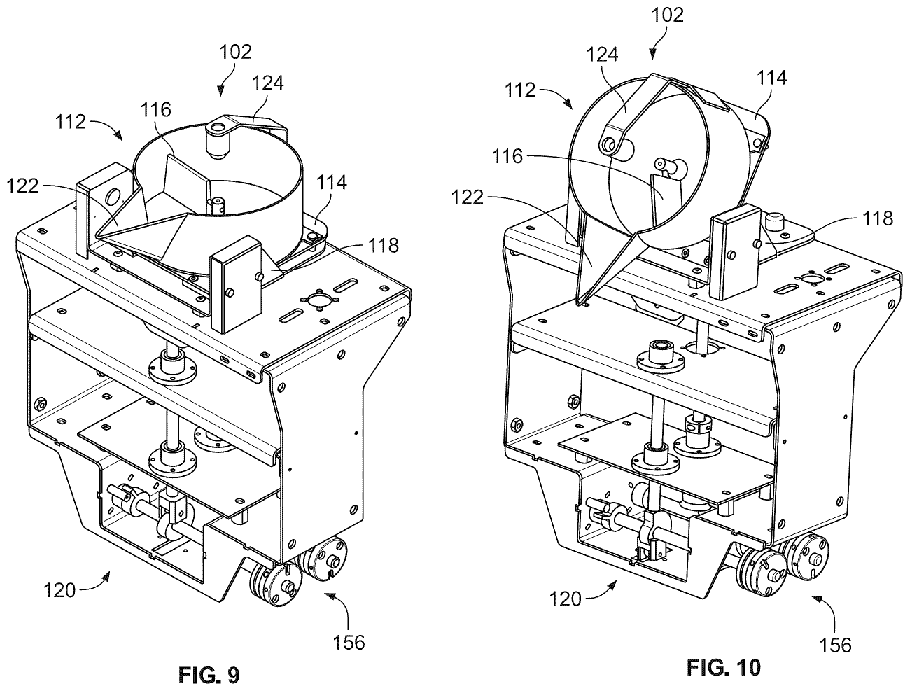

The tray 114 of the sterilization station 102 is configured to move (e.g., rotate, pivot, etc.) relative to the support structure 108 (via a pivot 118). In doing so, the sterilization reservoir 112 is configured to move with the tray 114. In the illustrated embodiment, the tray 114 (and reservoir 112) are configured to move between a first position (FIGS. 6 and 9) (e.g., a starting position, etc.), in which the tray 114 is oriented generally horizontally and the sterilization reservoir 112 is positioned generally flat, and a second position (FIGS. 7 and 10) (e.g., a dispensing position, etc.) in which the tray 114 is oriented at an angle relative to the support structure 108 and the sterilization reservoir 112 is generally tilted (or tipped). For example, as will be described more hereinafter, once the explant material is sterilized in the reservoir 112, an actuator assembly 120 (e.g., a motor and piston, etc.) is configured (via the computing device 110) to pivot the tray 114 generally toward a spout 122 of the sterilization reservoir 112 and move the sterilization reservoir 112 from the first position to the second position. In turn, the explant material and sterilization media in the sterilization reservoir 112 are directed to the spout 122 and are transferred, via the spout 122 (e.g., poured via gravity, etc.), to the rinse and floatation station 104.

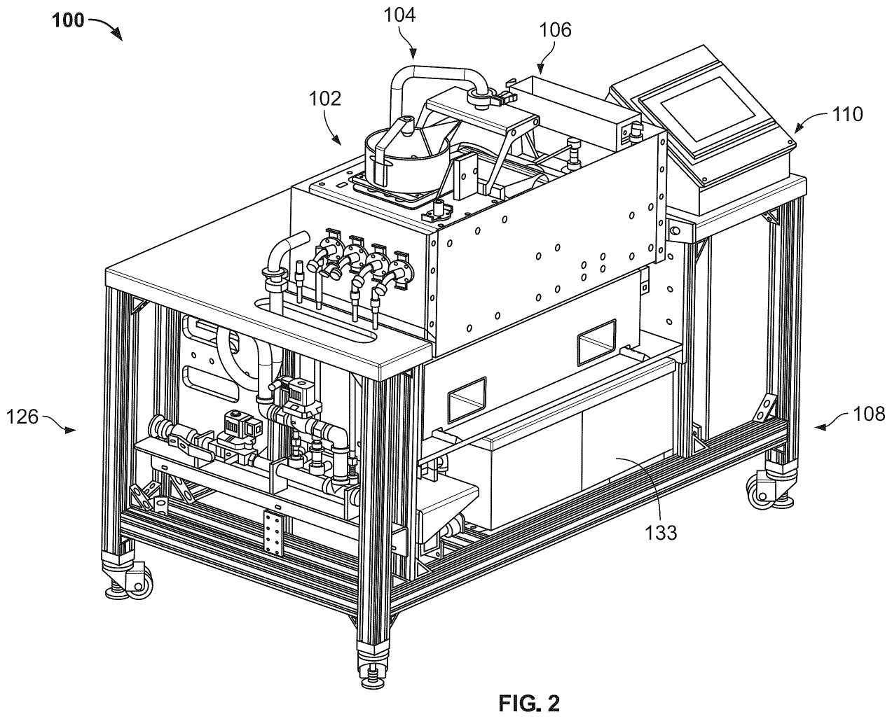

The sterilization station 102 also includes a fluid discharge 124 (e.g., a showerhead, a fluid discharge head, etc.) disposed generally over the sterilization reservoir 112 (e.g., coupled to the sterilization reservoir 112 and/or the tray 114, etc.). The fluid discharge 124 is configured to deliver fluid (e.g., sterile water, etc.) to the sterilization reservoir 112, for example, to rinse the explant material after sterilization, to provide fluid to help flow or carry the explant material from the sterilization reservoir 112 to the rinse and floatation station 104 (via gravity), etc. In connection therewith, the fluid discharge 124 is coupled in fluid communication to a fluid distribution unit 126 (FIGS. 2-4) disposed on the support structure 108 generally below (e.g., generally lower than, etc.) the sterilization station 102. The fluid distribution unit 126 includes a supply of fluid (e.g., sterile water, etc.) and is configured to provide the fluid, from the supply, to the fluid discharge 124 as needed (e.g., via one or more tubes, valves, pumps, etc.) (e.g., as directed or controlled by the computing device 110, etc.).

With reference now to FIGS. 11-13, the rinse and floatation station 104 includes a float tank 128 (or float chamber) configured to receive the sterilized explant material and sterilization media from the sterilization reservoir 112 of the sterilization station 102 (via the spout 122). In connection therewith, a filter 130 (e.g., a sieve, a grate, etc.) is positioned generally within the float tank 128, over a bottom portion of the tank, to collect the explant material transferred to the tank 128 (and to allow the sterilization media to pass through the filter 130 to the bottom portion of the tank 128). A drain 132 (FIG. 13) is located in the bottom portion of the float tank 128, generally under the filter 130 (e.g., in a bottom wall of the float tank 128, etc.). The drain 132 is configured to open (e.g., as controlled by the computing device 110, etc.) to drain the sterilization media and other fluid from the float tank 128 (e.g., that pass through the filter 130, etc.), as desired, for receipt/collection (e.g., via gravity, etc.) in a collection tank 133 (or wastewater/waste fluid tank) disposed on the support structure 108. In this manner, as the explant material and sterilization media are received in the float tank 128 from the sterilization reservoir 112 of the sterilization station 102, the explant material is captured on the filter 130 while the sterilization media passes through the filter 130 and is drained (or removed) from the float tank 128 via the drain 132 (and collected for subsequent processing, disposal, etc. in the collection tank 133 (which may be removed from the support structure 108 to allow for such disposal, etc.)).

The rinse and floatation station 104 also includes a fluid discharge 134 (e.g., a showerhead, etc.) disposed generally over the float tank 128 (e.g., coupled to the support structure 108, etc.). The fluid discharge 134 is configured to deliver fluid (e.g., sterile water, etc.) to the float tank 128, for example, to help further rinse the explant material after sterilization. In connection therewith, the fluid discharge 134 is coupled in fluid communication to the fluid distribution unit 126 disposed on the support structure 108. As such, as needed, the fluid distribution unit 126 is configured to provide the fluid, from the supply, to the fluid discharge 134 (e.g., via one or more tubes, valves, pumps, etc.) (e.g., as directed or controlled by the computing device 110, etc.) for use in rinsing the explant material in the float tank 128.

In addition in this embodiment, the float tank 128 of the rinse and floatation station 104 includes discharge ports 136 (e.g., bottom ports, etc.) located in the bottom portion of the float tank 128 (e.g., positioned in and/or coupled to the bottom wall of the float tank 128, etc.), and fluid jets 138 disposed in a rearward portion of the float tank 128 (e.g., positioned in a rearward wall or sidewall of the float tank 128, etc.). The discharge ports 136 and fluid jets 138 are configured to deliver fluid (e.g., sterile water, etc.) to the float tank 128, for example, to facilitate movement (e.g., floating, etc.) of the explant material from within the float tank 128 (e.g., from the filter 130 within the float tank 128, etc.) to a discharge 140 of the float tank 128 (e.g., an elevated ledge of the float tank 128, a weir, etc.), which in turn directs the explant material to the rehydration station 106. In connection therewith, the discharge ports 136 and the fluid jets 138 are coupled in fluid communication to the fluid distribution unit 126 disposed on the support structure 108 (via one or more manifolds, etc.). As such, as needed, the fluid distribution unit 126 is configured to provide the fluid, from the supply, to the discharge ports 136 and the fluid jets 138 (e.g., via one or more tubes, valves, pumps, etc.) (e.g., as directed or controlled by the computing device 110, etc.) for use as described herein. For instance, once the sterilization media is drained from the float tank 128, and the explant material is rinsed via the discharge 134 (and the rinse fluid is drained), the drain 132 within the float tank 128 is closed (e.g., via the computing device 110, etc.) and fluid is directed to the discharge ports 136. As the fluid enters and fills the float tank 128, via the discharge ports 136, the explant material raises off the filter 130 (e.g., floats, etc.). And, as the fluid level in the float tank 128 approaches an upper portion of the tank 128, the fluid jets 138 are activated (e.g., by the computing device 110, etc.) to thereby discharge fluid into the float tank 128 and direct the floating explant material to the discharge 140 (via directional flow/eddies caused by the jets 138, etc.).

In the illustrated embodiment, the float tank 128 of the rinse and floatation station 104 includes four discharge ports 136 located in the bottom portion of the tank 128. It should be appreciated, though, that the float tank 128 may include any desired number of ports within the scope of the present disclosure (e.g., at least one port, one port, two ports, three portions, four ports, six ports, more than six ports, etc.). Similarly, it should be appreciated that the float tank 128 may include any desired number of jets within the scope of the present disclosure (e.g., at least one jet, two jets, four jets, ten jets, more than ten jets, an array of jets, etc.). In addition, in the illustrated embodiment, the fluid jets 138 are positioned in the back portion of the float tank 128 at an elevation in the tank 128 generally higher than the discharge 140. The fluid jets 138, then, are directed generally at the discharge 140 (e.g., at a bottom part of a curve defined by the discharge 140, etc.) to thereby create a desired directional flow and/or eddies for moving the explant material out of the float tank 128.

In example embodiments, the rinse and floatation station 104 (and more generally, the floatation operation herein) may be configured to separate more transformable explants from less transformable explants.

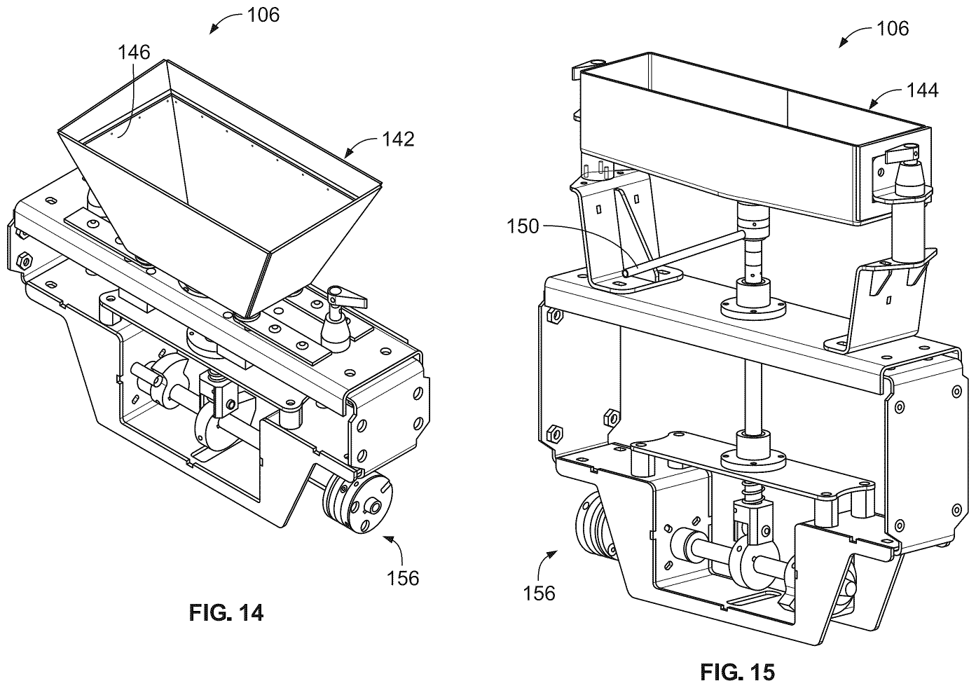

Referring now to FIGS. 14-16, the rehydration station 106 includes a rehydration tank 142 and a rehydration fluid storage chamber 144. The rehydration tank 142 is configured to receive the rinsed explant material from the float tank 128 of the rinse and floatation station 104 (e.g., from the discharge 140 of the tank 128, etc.). In connection therewith, a filter 146 (e.g., a sieve, a grate, etc.) is positioned generally within the rehydration tank 142, over a bottom portion of the rehydration tank 142, to collect the explant material transferred to the rehydration tank 142 (and to allow the fluid received from the float tank 128 with the explant material to pass through the filter 146 to the bottom portion of the rehydration tank 142). A drain 148 is located in the bottom portion of the rehydration tank 142, generally under the filter 146 (e.g., in a bottom wall of the rehydration tank 142, etc.). The drain 148 is configured to open (as instructed by the computing device 110, etc.) and drain the fluid from the rehydration tank 142 (e.g., that passes through the filter 146, etc.), as desired, for receipt in the collection tank 133. In this manner, as the explant material and fluid are received in the rehydration tank 142 from the float tank 128 of the rinse and floatation station 104, the explant material is captured on the filter 146 while the fluid passes through the filter 146 and is drained (or removed) from the rehydration tank 142 via the drain 148 (and collected for subsequent processing, disposal, etc. in the collection tank 133).

The rehydration fluid storage chamber 144 is configured to deliver rehydration fluid to the rehydration tank 142 (and the explant material received therein), for example, once the fluid received from the float tank 128 of the rinse and floatation station 104 is drained. In connection therewith, the rehydration fluid storage chamber 144 includes a spout 150 coupled in fluid communication with the rehydration fluid storage chamber 144. The spout 150, then, is configured to direct the rehydration fluid from the rehydration fluid storage chamber 144 and into the rehydration tank 142 (e.g., via control of one or more valves, etc. by the computing device 110; etc.), for use in rehydration of the explant material captured on the filter 146.

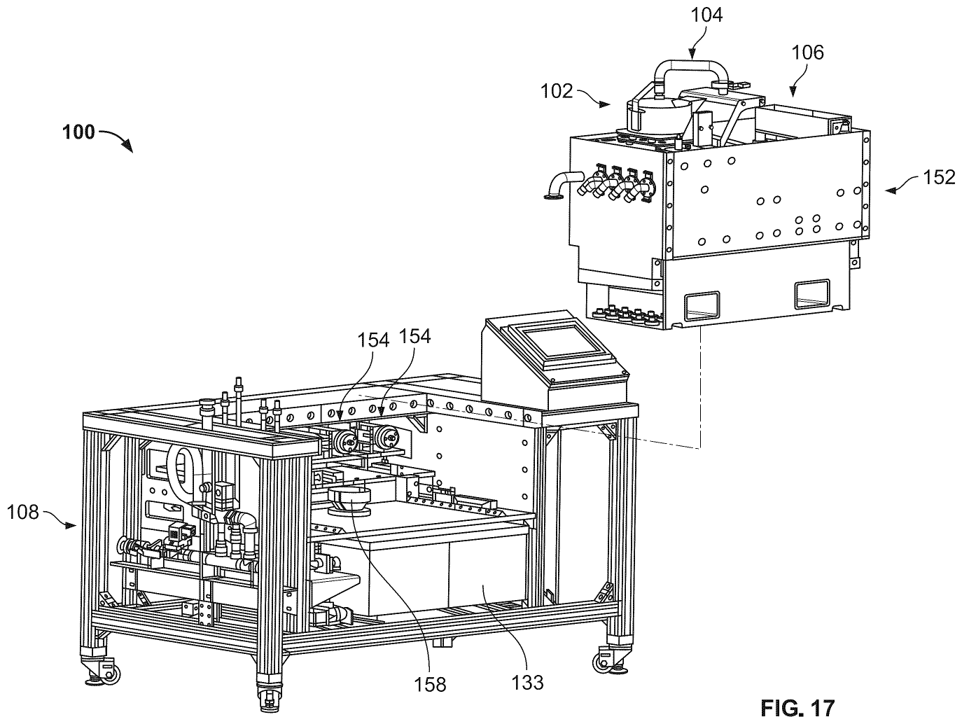

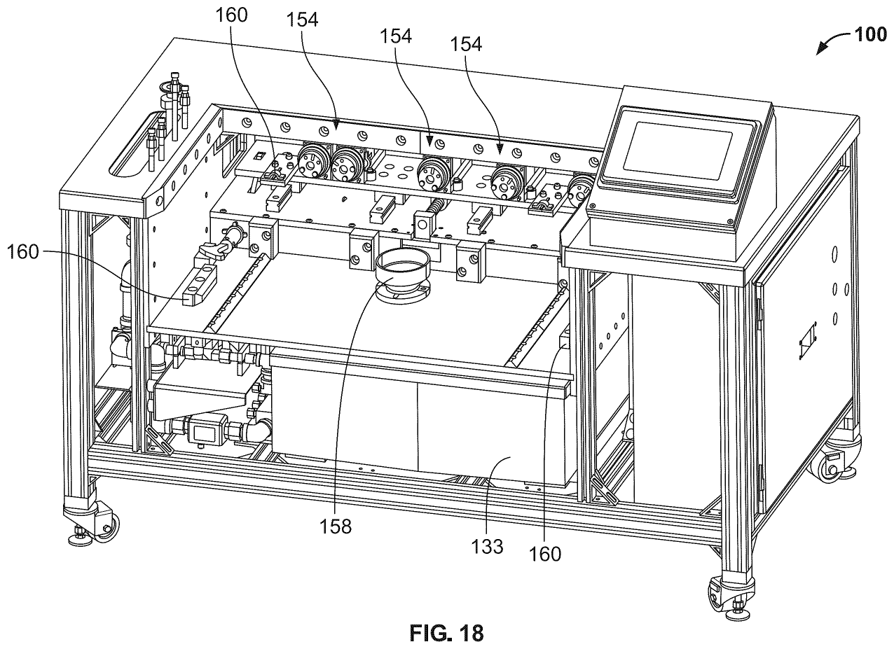

With reference now to FIGS. 17 and 18, in the illustrated embodiment, the sterilization station 102, the rinse and floatation station 104, and the rehydration station 106 are each included in a removable module 152 (e.g., a containment box, etc.) of the system 100. In this way, the sterilization station 102, the rinse and floatation station 104, and the rehydration station 106 can be removed from the system, as part of the module 152, as needed (e.g., for autoclaving, maintenance, etc.). In connection therewith, the sterilization station 102, the rinse and floatation station 104, and the rehydration station 106 are arranged in the module 152 so that, when positioned in the support structure 108 (e.g., within a nest of the support structure 108, etc.), drive motors 154 of the system readily align with corresponding motor components 156 of the sterilization station 102, the rinse and floatation station 104, and the rehydration station 106 for operation (e.g., cam shafts align for actuating the various parts of the stations as described herein, etc.). For instance, lugs associated with the drive motors 154 and corresponding motor components 156 are configured to engage when the module 152 is installed in the support structure 108 (e.g., within the nest of the support structure, etc.), to allow for the drive motors 154 to operate the motor components 156, and then disengage when the module is removed 152. In addition, the drains 132, 148 of the float tank 128 and the rehydration tank 142 are configured (within the module 152) to direct the fluid removed from the thanks 128, 148 to a main collection drain 158 within the support structure 108 for directing the fluid from the tanks 128, 148 to the collection tank 133. Further, sensors 160 may be included in the support structure 108 to confirm correct alignment of the motor components 156 and/or drains when the module 152 is installed in the support structure 108. Additionally, or alternatively, sensors may be included on the module 152 to similarly help confirm such correct alignment.

An example method of preparing explant material for subsequent use, via the system 100, will be described next.

At the outset in this example method, the system 100 is prepared, or initialized, to receive and process the explant material. Table 1 includes example operations that may be performed (e.g., in an automated manner via the computing device 110, etc.) as part of the initialization process, at various stages thereof, and example parameters that may lead to the described action. In the example process of Table 1, initiation variable “None” indicates a starting or continued action without prompt, and initiation variable “NA” indicates that completion of a previous stage acts as an automatic prompt for the given stage.

TABLE 1

| | | Initiation | | |
|---|---|---|---|---|
| Stage | Component | Variable | Action | Parameter |
| A. | SYSTEM (100) | None | Operator Select | — |
| | | | PURGE | |
| | | | SYSTEM | |
| | | | (AUTOMATIC) | |
| B. | BUTTON (VIA COMPUTING | NA | Operator press | — |
| | DEVICE 110) | | START | |
| C. | ALL DRAINS (132, 148) | NA | OPEN | — |
| | ALL VALVES | NA | CLOSED | — |
| D. | FLOAT TANK (128) AND | NA | OPEN | — |
| | VALVES (LEFT PORTS) & | | | |
| | (RIGHT PORTS) | | | |
| | DIRECTIONAL PORT | NA | OPEN | — |
| | VALVE | | | |
| | STERILIZATION | NA | OPEN | — |
| | SHOWERHEAD VALVE | | | |
| | RINSE SHOWERHEAD | NA | OPEN | — |
| | VALVE | | | |
| E. | STERILIZATION TRAY | NA | TILT | — |
| | (114) | | | |
| F. | MAIN VALVE | NA | OPEN | Time Variable |
| | | | | determined by |
| | | | | number of |
| | | | | explants |
| | | | | processed |
| G. | | Not enough | ALARM & | New cycle on |
| | | pressure or | COMPLETION | hold until |
| | | flow rate | OF CYCLE | manual |
| | | | | clearance of |
| | | | | alarm condition |
| H. | CRITICAL LOW RANGE | NA | ALARM & STOP | Time: 3 |
| | ALARM | | | seconds |
| I. | CRITICAL HIGH RANGE | NA | ALARM & STOP | Time: 3 |
| | ALARM | | | seconds |
| J. | ALL VALVES | NA | CLOSE | — |
| | ALL DRAINS | NA | OPEN | — |

Once the system 100 is initiated (e.g., once the initiation process is complete, etc.), explant material (e.g., weighed and/or counted excised explant material, etc.) and sterilization media are added to the sterilization reservoir 112 of the sterilization station 102 (e.g., manually, via an automated delivery system, etc.). The explant material may include excised explant material removed from frozen storage and thawed (e.g., thawed for a desired amount of time (e.g., about 30 minutes or more or less, etc.), etc.), or it may include excised explant material directly removed from desired plants (without being first stored). In either case, the explant material may include any desired number of explants, for example, between about 6,000 and about 75,000 explants (or more or less). And, the sterilization media may include any desired sterilization media (e.g., a media comprising about 70% ethanol containing 100 g/L polyethylene glycol (PEG MW 8000), etc.). At about the same time, or before or after, in this example, rehydration media is also added to the rehydration fluid storage chamber 144 (as needed) (e.g., manually, automatically via an automated fluid delivery system in communication with the storage chamber 144, etc.). And, an automated sterilize cycle begins (e.g., as initiated by or via the computing device 110, etc.). That said, in some examples, the rehydration media added to the rehydration fluid storage chamber 144 may be pre-heated (e.g., based on the heating parameters described above, etc.). Or, as described above, a heating element may be functionally connected to the rehydration station 106 (e.g. at the rehydration fluid storage chamber 144, etc.) to head the rehydration media as described above.

During the sterilize cycle, the paddle 116 of the sterilization station 102 oscillates (as controlled by the computing device 110) the mixture of explant material and sterilization media within the sterilization reservoir 112 (e.g., at a desired rate of the paddle 116 and for a desired time, etc.). Next, the tray 114 of the sterilization station 102 automatically tilts upward (e.g., with gradually increasing tilt speed, etc.) and thereby directs the contents (e.g., explant material and sterilization media, etc.) from the sterilization reservoir 112 to the spout 122 for delivery, via the spout 122, into the float tank 128 of the rinse and floatation station 104. After about half of the contents of the sterilization reservoir 112 have been transferred to the float tank 128 of the rinse and floatation station 104, the fluid discharge 124 of the sterilization station 102 initiates (as controlled by the computing device 110) and discharges fluid (e.g., sterile water as supplied by the fluid distribution unit 126, etc.) into the sterilization reservoir 112, as the tray 114 continues to tilt the reservoir 112 (see, e.g., FIG. 7, etc.).

When the tilt of the sterilization reservoir 112 (and tray 114) is complete, the tray 114 lowers the reservoir 112 back to its starting position (e.g., with the tray 114 generally horizontal, etc.). In connection therewith, the fluid discharge 124 of the sterilization station 102 continues to deliver fluid into the sterilization reservoir 112. Then, when a desired amount of fluid is received in the sterilization reservoir 112, the tray 114 again tilts the reservoir 112 toward the rinse and floatation station 104, in substantially the same manner previously described, and thereby directs the remaining contents (e.g., any remaining excised explants, fluid, and sterilization media; etc.) from the reservoir to the spout 122 for delivery into the float tank 128 of the rinse and floatation station 104. When the tray 114 reaches the fully tilted position, again, the fluid discharge 124 is turned off (via the computing device 110, etc.). And, the sterilization reservoir 112 returns to the starting, un-tilted, position, thereby completing the sterilize cycle.

Table 2 includes example operations that may be performed (e.g., in an automated manner via the computing device 110, etc.) as part of the sterilize cycle/process, at various stages thereof. In the example process of Table 2, again, the initiation variable “None” indicates a starting or continued action without prompt, and the initiation variable “NA” indicates that completion of a previous stage acts as an automatic prompt for the given stage. In addition, example batch sizes of explants that may be processed by the are system 100 are referenced in Table 2, including a small batch size (for example, having between about 6,000 and about 35,999 explants, etc.) and a large batch size (for example, having between about 36,000 and about 75,000 explants, etc.).

TABLE 2

| | | Initiation | | | |
|---|---|---|---|---|---|
| Stage | Component | Variable | Action | | Time |
| 1.1 | — | None | Operator THAW selected | | 30 min to |
| | | | amount of excised | | THAW |
| | | | explants | | |
| 1.2 | — | NA | Operator ADD explants | | — |
| | | | to sterilization reservoir (112) | | |
| | | | Small | Large | |
| | | | Batch: | Batch: | |
| | | | 6,000- | 36,000- | |
| | | | 35,999 | 80,000 | |
| | | | explants | explants | |
| 1.3 | — | NA | Operator ADD | | — |
| | | | sterilization media to | | |
| | | | sterilization reservoir (112) | | |
| | | | Small | Large | |
| | | | Batch: 600 mL | Batch: 1,100 mL | |
| | | | sterilization | sterilization | |
| | | | media | media | |
| 1.4 | SYSTEM (100) | NA | Operator SELECT | | — |
| | | | “STERILIZE” (via | | |
| | | | computing device 110) | | |
| 1.5 | BUTTON (VIA | NA | Operator PRESS START | | — |
| | COMPUTING | | | | |
| | DEVICE 110) | | | | |
| 1.6 | STERILIZATION | NA | OSCILLATE mixture of | | 3 min 30 sec |
| | TRAY (114)/ | | explants and sterilization | | OSCILLATION |
| | OSCILLATING | | media at 60 RPM (via | | |
| | PADDLE (116) | | computing device 110) | | |
| 1.7 | STERILIZATION | NA | TILT UPWARD: | | ~15 sec to TILT |
| | TRAY (114) | | gradually increasing | | |
| | | | speed (via computing | | |
| | | | device 110) | | |
| | | | Tray contents pour to | | |
| | | | float tank (128) | | |
| 1.8 | STERILIZATION | 7.5 sec into | Turn ON (via | | — |
| | SHOWERHEAD (124) | STAGE 1.7 | computing device 110) | | |
| 1.9 | STERILIZATION | Tilt | LOWER (TILT | | ~15 sec to |
| | TRAY (114) | completed | REDUCED) (via | | LOWER |
| | | | computing device 110) | | |
| — | STERILIZATION | None | REMAINS ON | | — |
| | SHOWERHEAD (124) | | | | |

| | | | | |
|---|---|---|---|---|
| 1.10 | STERILIZATION | Filled to | TILT UPWARD: | ~15 sec to TILT |
| | TRAY (114) | ~1,100 mL | gradually increasing | |
| | | with sterile | speed (via computing | |
| | | water | device 110) | |
| | | | Tray contents pour to | |
| | | | float tank (128) | |
| 1.11 | STERILIZATION | Sterilization | Turn OFF (via | — |
| | SHOWERHEAD (124) | tray 114 | computing device 110) | |

reaches full
tilted
position

| | | | | |
|---|---|---|---|---|
| 1.12 | STERILIZATION | NA | LOWER (TILT | — |
| | TRAY (114) | | REDUCED) (via | |
| | | | computing device 110) | |

That said, it should be appreciated that other categories of batch sizes for explants may be provided/used in other example embodiments (other than illustrated in Table 2). For example, the categories may include a small batch size (having between about 6.000 and about 14,999 explants), a medium batch size (having between about 15,000 and about 34,999 explants), and a large batch size (having between about 35,000 and about 80,000 explants). In such instances, at Stage 1.3, the same amount of sterilization media will be used for both the medium batch size and the large batch size (e.g., 1,100 mL, etc.). In addition, it should be appreciated that other numbers of explants may be included in other batch sizes within the scope of the present disclosure. For instance, the large batch size may have upwards of 100,000 explants in some embodiments.

In connection with the above, a rinse cycle is automatically initiated (at the rinse and floatation station 104), via the computing device 110, following sterilization of the explant material (e.g., following completion of the sterilize cycle, etc.). Here, as the sterilized explant material and sterilization media is delivered to the float tank 128 of the rinse and floatation station 104 (and the explant material is received on the filter 130 within the tank 128), the drain 132 of the float tank 128 is opened (by the computing device 110, etc.) such that the sterilization media drains from the float tank 128 and leaves the sterilized explant material in/on the filter 130 (within the tank 128). Once all of the explant material is received from the sterilization station 102, the fluid discharge 134 of the rinse and floatation station 104 is initiated (via the computing device 110) and directs fluid onto the explant material on the filter 130 (e.g., sterile water as supplied by the fluid distribution unit 126, etc.). After the appropriate time for the given batch size of explant material, the fluid discharge 134 is turned off, and the rinse cycle is complete.

Table 3 includes example operations that may be performed (e.g., in an automated manner via the computing device 110, etc.) as part of the rinse cycle/process, at various stages thereof. In the example process of Table 3, again, the initiation variable “None” indicates a starting or continued action without prompt, and the initiation variable “NA” indicates that completion of a previous stage acts as an automatic prompt for the given stage. In addition, example batch sizes of explants that may be processed by the are system 100 are referenced in Table 3, including a small batch size (for example, having between about 6,000 and about 35,999 explants, etc.) and a large batch size (for example, having between about 36,000 and about 75,000 explants, etc.).

TABLE 3

| | | Initiation | | | |
|---|---|---|---|---|---|
| Stage | Component | Variable | Action | Time | |
| — | — | Automatic | RINSE cycle | — | |
| | | continuation after | begins | | |
| | | completion of | | | |
| | | STERILIZATION | | | |
| | | cycle | | | |
| 2.1 | FLOAT TANK DRAIN | Completion of | OPEN (via | — | |
| | (132) | STERILIZATION | computing | | |
| | | cycle | device 110) | | |
| 2.2 | RINSE AND | NA | Turn ON: flow | Small | Large |
| | FLOATATION | | rate ~15 L/min | Batch: | Batch: |
| | SHOWERHEAD (134) | | (via computing | 30 sec | 60 sec |
| | | | device 110) | | |
| 2.3 | RINSE AND | Time appropriate | Turn OFF (via | — | |
| | FLOATATION | for batch size | computing | | |
| | SHOWERHEAD (134) | passes | device 110) | | |

That said, it should again be appreciated that other categories of batch sizes for explants may be provided/used in other example embodiments (other than illustrated in Table 3). For example, the categories may include a small batch size (having between about 6,000 and about 14,999 explants), a medium batch size (having between about 15,000 and about 34,999 explants), and a large batch size (having between about 35,000 and about 80,000 explants). In such instances, at Stage 2.2, the same rinse time may be used for both the small batch size and the medium batch size (e.g., 20 sec, etc.).

After completion of the rinse cycle, the computing device 110 automatically initiates a float cycle. At this cycle, the sterilized and rinsed explant material is located within the filter 130 of the float tank 128 of the rinse and floatation station 104 (following completion of the rinse cycle), and the drain 132 thereof is closed. Then, the bottom discharge ports 136 within the float tank 128 are activated (via the computing device 110) and the tank 128 is filled with fluid (e.g., sterile water as supplied by the fluid distribution unit 126, etc.) from the ports 136. Once the float tank 128 is filled with a desired amount of fluid, via the discharge ports 136, the ports 136 are turned off (via the computing device 110) and the explant material is floated via the fluid in the float tank 128 in a rested/suspended state for a desired amount of time. After the desired float time, the discharge ports 136 and the fluid jets 138 are activated for another desired period of time, agitating the fluid in the float tank 128 such that the floating (viable/non-debris) explant material flows over the discharge 140 (e.g., elevated ledge, etc.) of the float tank 128 and into the rehydration tank 142 of the rehydration station 106 (specifically, into the filter 146 located within the rehydration tank 142). During this time, the drain 132 in the float tank 128 quickly opens and closes to release water tension at a surface of the float tank 128 and causes more explant material to flow over the discharge 140. Then, the drain 132 is opened to drain the remaining fluid from the float tank 128. In some example embodiments, the above operations may be repeated one more times (e.g., to help ensure all explant material is removed/floated from the float tank 128, etc.), and the float cycle is then complete. That said, in some embodiments, one or more additional float cycles may be triggered by an operator (via an input to the computing device 110, etc.), for instance, to add more float cycles manually (e.g., as needed or desired based on explant type, inspection, etc.), whereby the manually triggered float cycle may or may not then be repeated.

Table 4 includes example operations that may be performed (e.g., in an automated manner via the computing device 110, etc.) as part of the float cycle/process, at various stages thereof. In the example process of Table 4, again, the initiation variable “None” indicates a starting or continued action without prompt and the initiation variable “NA” indicates that completion of a previous stage acts as a prompt for the stage.

TABLE 4

| | | Initiation | | |
|---|---|---|---|---|
| Stage | Component | Variable | Action | Time |
| — | — | Automatic | FLOAT cycle | — |
| | | continuation after | begins | |
| | | completion of | | |
| | | RINSE cycle | | |
| 3.1 | FLOAT TANK | Completion of | CLOSE (via | — |
| | DRAIN (132) | RINSE cycle | computing device | |
| | | | 110) | |
| 3.2 | BOTTOM PORTS | NA | Turn ON (via | ~8 sec to FILL |
| | (136) | | computing device | chamber |
| | | | 110) | |
| 3.3 | BOTTOM PORTS | NA | Turn OFF (via | ~20 sec FLOAT |
| | (136) | | computing device | |
| | | | 110) | |
| 3.4 | BOTTOM PORTS | NA | Turn ON (via | ~10 sec |
| | (136) | | computing device | AGITATION |
| | | | 110) | and FLOW |
| | REAR UPPER JET(S) | Water reaches | Turn ON (via | |
| | (138) | surface level of | computing device | |
| | | Float Tank (128) | 110) | |
| | FLOAT TANK | NA | OPEN and | |
| | DRAIN (132) | | CLOSE quickly | |
| | | | (release water | |
| | | | tension) (via | |
| | | | computing device | |
| | | | 110) | |
| 3.5 | FLOAT TANK | NA | OPEN (via | ~8 sec to |
| | DRAIN (132) | | computing device | DRAIN |
| | | | 110) | chamber |
| 3.6 | — | NA | REPEAT Stages | — |
| | | | 3.1-3.5 ONE time | |

In connection with the above, as the sterilized, rinsed, and floated explant material is received in the rehydration tank 142 of the rehydration station 106 (as part of the float cycle), the drain 148 of the rehydration tank 142 is opened (via the computing device 110). As such, the explant material is received in the rehydration tank 142 on the filter 146 (and maintained in the tank 142 by the filter 146), and any fluid received in the rehydration tank 142 with the explant material is removed through the drain 148. Then, once the float cycle is completed (and all of the explant material is transferred to the rehydration station 106), the computing device 110 automatically initiates a rehydration cycle.

In the rehydration cycle, the drain 148 of the rehydration tank 142 is closed (via operation of the computing device 110). Then, rehydration (or hydration) media from the rehydration fluid storage chamber 144 is delivered to the rehydration tank 142 through the spout 150 (via operation of one or more valves, etc.). The rehydration media may include any desired rehydration media (e.g., a media comprising ⅖ strength B5 macro salts except ½ strength CaCl₂), 1/10 strength B5 vitamins and micro salts, 2.8 mg/L sequestrene, 1 g/L potassium nitrate, 30 g/L dextrose, 3.9 g/L MES, 0.03 g/L Clearys 3336 WP, pH 5.4; etc.). The explant material is soaked in the rehydration media, within the rehydration tank 142, for a desired amount of time. Upon completion of the allotted soak time, the drain 148 of the rehydration tank 142 is opened (via the computing device 110, etc.) and the rehydration media is drained from the tank 142. The explant material may then be removed from the rehydration tank 142, via the filter 146, etc., for subsequent use. Or, the rehydration tank 142 may be removed all together from the system 100 (e.g., from the support structure 108, etc.) to thereby allow for collection of the explant material. In either case, the collected explant material may be used in a normal transformation process, applicable for use on the excised explant material. And, the method may be restarted for another batch of explant material.

Table 5 includes example operations that may be performed (e.g., in an automated manner via the computing device 110, etc.) as part of the rehydration cycle/process, at various stages thereof. In the example process of Table 5, again, the initiation variable “None” indicates a starting or continued action without prompt, and the initiation variable “NA” indicates that completion of a previous stage acts as an automatic prompt for the given stage. In addition, example batch sizes of explants that may be processed by the are system 100 are referenced in Table 3, including a small batch size (for example, having between about 6,000 and about 35,999 explants, etc.) and a large batch size (for example, having between about 36,000 and about 75,000 explants, etc.).

TABLE 5

| | | Initiation | | |
|---|---|---|---|---|
| Stage | Component | Variable | Action | Time |
| — | — | Automatic | REHYDRATE cycle | — |
| | | continuation | begins | |
| | | after | | |
| | | completion of | | |
| | | FLOAT cycle | | |
| 4.1 | REHYDRATION | Completion of | CLOSE (via computing | — |
| | TANK DRAIN (148) | FLOAT cycle | device 110) | |
| 4.2 | HYDRATION | NA | OPEN (via computing | ~XX sec to |
| | FLUID | | device 110) | DRAIN |
| | CONTAINER | | | rehydration |
| | VALVE | | | media tank |

| | | | | |
|---|---|---|---|---|
| 4.3 | — | NA | SOAK | ~1-2 hours |

| | |
|---|---|
| Small | Large |
| Batch: | Batch: |
| 600 mL | 1,000 mL |
| rehydration | rehydration |
| media | media |

| | | | | |
|---|---|---|---|---|
| 4.4 | REHYDRATION | Completion of | OPEN (via computing | — |
| | TANK DRAIN (148) | SOAK time | device 110) | |

That said, it should again be appreciated that other categories of batch sizes for explants may be provided/used in other example embodiments (other than illustrated in Table 5). For example, the categories may include a small batch size (having between about 6,000 and about 14,999 explants), a medium batch size (having between about 15,000 and about 34,999 explants), and a large batch size (having between about 35,000 and about 80,000 explants). In such instances, then, at Stage 4.3, for example, 1,000 mL of rehydration media may be used for the medium batch size and 1,600 mL of rehydration media may be used for the large batch size.

In some example embodiments, the above automated method may proceed as described only through the float cycle, where the explant material is delivered to the rehydration tank 142 of the rehydration station 106. In such embodiments, then, the rehydration tank 142 may be removed from the system 100 prior to introduction of the rehydration media from the rehydration fluid storage chamber 144. Rehydration of the explant material may then be performed manually on the explant material, in the rehydration tank 142 apart from the system 100, while the method may restart to process another batch of explant material, for example, with another rehydration tank inserted into the system 100 (e.g., positioned on the support structure, etc.) in place of the rehydration tank 142 removed. As can be appreciated, this operation of the system 100 may allow for processing additional batches of explant material within the system 100, without waiting for the rehydration of the explant material (at the rehydration station 106) to complete before another batch of explant material can be processed (particularly since the rehydration cycle requires the longest amount of time in the method (see, e.g., Table 5, etc.).

### EXAMPLES

The following examples are exemplary in nature. Variations of the following example are possible without departing from the scope of the disclosure.

### Example 1

In this example, excised explants (e.g., explant material) were sterilized via the automated method described above (using the system 100) and compared to explants sterilized via a conventional manual process. Viability of the explants was then compared. As will be shown, the viability of the explants sterilized via the automated method above was not adversely affected, as compared to the explants sterilized via the conventional manual process.

In two separate trials, the two treatments (Automated Sterilization treatment and Manual Sterilization treatment) were initiated side-by-side. The Automated Sterilization treatment was carried out as in Table 2 above. The Manual Sterilization treatment included about 6,840 explants being sterilized in a 1 L roller bottle containing 600 mL of sterilizing solution (70% ethanol containing 100 g/L PEG MW 8000 (polyethylene glycol)) for 3 minutes and 30 seconds. The Automated Sterilization treatment was followed by the Automated Rinse and Automated Floatation cycles as described in Example 2 (and in Tables 3 and 4 above). The Manual Sterilization treatment was followed by Manual Rinse and Manual Floatation treatments. In such manual treatments, the explants were manually rinsed with autoclaved water by pouring water into the roller bottle, agitating the bottle to agitate the explants, and straining off the water and repeating for a total of 5 rinses. After the rinsing, the manual floatation method was performed by adding 500 mL of sterile water into the roller bottle or a beaker and allowing explants to float to the surface. The floating explants were poured off into a strainer, leaving debris behind. The float process was repeated until no more viable explants floated off, and the remaining explant debris was discarded as waste.

After completion of the floatation trial comparisons, explants from each treatment were manually rehydrated in 200 mL of rehydration media comprising: ⅖ strength of B5 macro salts except ½ strength CaCl₂), 1/10 strength of B5 micro salts and vitamins, 1 g/L potassium nitrate, 30 g/L dextrose, 2.8 mg/L sequestrene, 3.9 g/L 2-(N-morpholino) ethanesulfonic acid (MES), and 0.03 g/L Clearys 3336 WP (pH 5.4), and sterile water for 1-2 hours with agitation on a shaker at about 80 RPM and regenerated through a standard regeneration assay to assay for viability. The standard regeneration assay began with rinsing the rehydrated explants from both treatment groups with sterile water and agitation for a total of 3 rinses. Then, the explants from both treatments were distributed across Plantcons containing 100 mL each of regeneration media comprising: 3.21 g/L Gamborgs B5 Medium, 20 g/L sucrose, 1.29 g/L Calcium Gloconate, 0.03 g/L Clearys 3336 WP, 4 g/L agargel, 0.8 mL Carbenicillin (250 mg/mL stock), 1 mL Timentin (100 mg/mL stock), 2 mL Cefotaxime (100 mg/mL stock), and sterile water. The Plantcons were incubated at 28+3° C. with a 16 hour light/8 hour dark photoperiod and light range of 100-180 pE for two weeks. After two weeks, viable explants, those with the presence of a growing point at the meristematic region of the embryo, were counted.

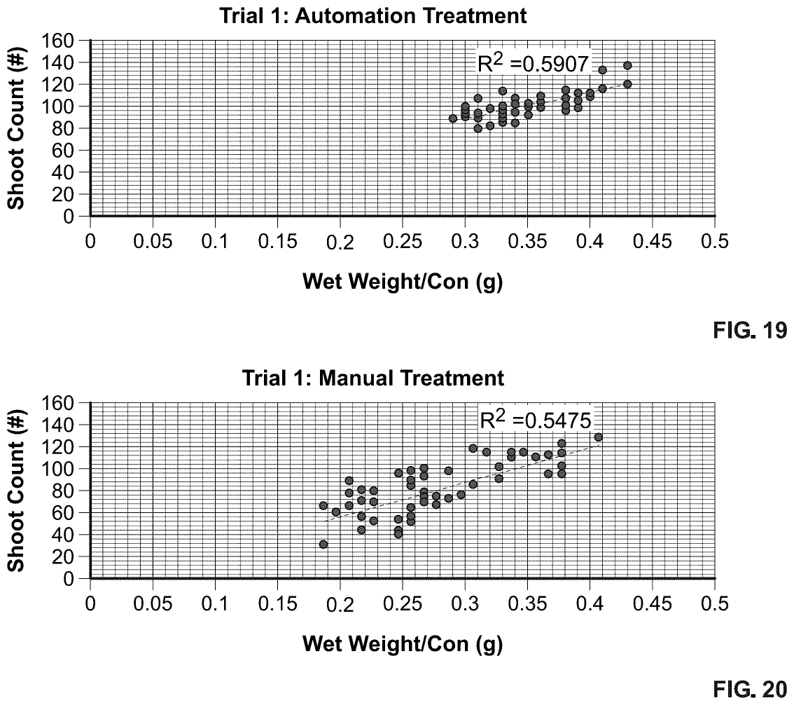

In two separate replicates (Trial 1 and Trial 2) of the abovementioned treatments (hereby referred to as “Manual” and “Automation”), the sterilization method comparison trials were carried out. The resulting viability was plotted using measurements of the number of shoots counted per regeneration container versus the wet weight of the container. The results are illustrated in FIGS. 19-22. Each graph depicts an x-axis denoting the wet weight of explants added to the regeneration container in grams. The y-axis denotes the number of shoots after the regeneration period. Each datapoint depicts a single regeneration container. The R² values represent the proportion of the difference between data points for Shoot Count (#)/Wet Weight (g). The R² values denote the variation within each dataset allowing for comparisons between the automated and manual treatments. As shown, there was less than 2% loss of plates to contamination in each treatment of Trial 1. In addition, there was about 9% loss of plates to contamination in the Automation Treatment and about 5% loss of plates to contamination in the Manual Treatment in Trial 2. This contamination increase relative to Trial 1 is attributed to use of a different batch of explants with a known higher bioburden.

The total counts and averages from the treatments are found in Tables 6 and 7. In the tables, “Treatment” refers to the treatment (Automation or Manual) applied. “Total Wet Weight (g)” refers to the total weight of prepared “wet” excised explants collected after rehydration. “Shoot Count” refers to the total number of shoots produced across all regeneration containers of the treatment. “Shoots/gram” is the Shoot Count divided by the Total Weight (g) per treatment. “Avg. Con weight/Total” is calculated by each individual regeneration container weight (g) divided by the Total Wet Weight (g) and then averaged across the treatment. “Avg. Con shoot #/Total” is calculated by each individual regeneration container number of shoots divided by the total Shoot Count and then averaged across the treatment.

TABLE 6

| | Total | Shoot | | Avg. Con | Avg. Con |
|---|---|---|---|---|---|
| Treatment | Weight (g) | Count | Shoots/gram | weight/Total | shoot #/Total |
| Automation | 17.96 | 5174 | 288.0846325 | 1.82% | 1.96% |
| (Trial 1) | | | | | |
| Manual | 16.54 | 4729 | 285.9129383 | 1.82% | 1.82% |
| (Trial 1) | | | | | |

TABLE 7

| | Total Wet | Shoot | | Avg. Con | Avg. Con |
|---|---|---|---|---|---|
| Treatment | Weight (g) | Count | Shoots/gram | weight/Total | shoot #/Total |
| Automation | 21.33 | 6790 | 318.3309892 | 2.14% | 3.36% |
| (Trial 2) | | | | | |
| Manual | 21.13 | 7012 | 331.8504496 | 2.32% | 3.80% |
| (Trial 2) | | | | | |

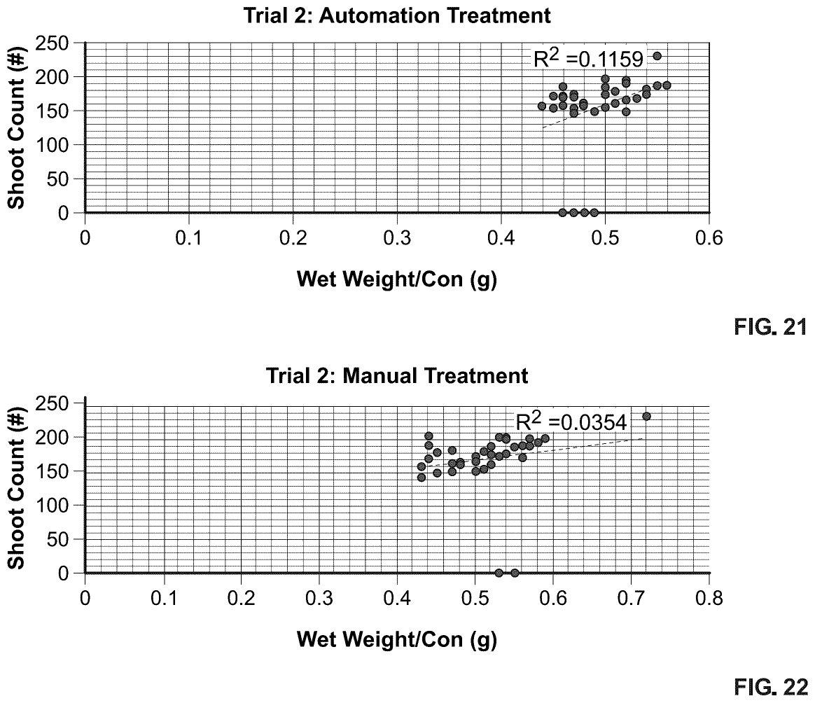

In Trial 1, both treatments showed similar levels of variability between data points (R²=0.5907 and R²=0.5475, FIGS. 19 and 20), indicating a reasonable comparison between the viability results of the treatments. The results of Trial 1 (Table 6 column “Avg. Con shoot #/Total”) indicate similar viability between the automated and manual treatments. In Trial 2, there was an increased difference in the R² values between the automated and variable treatments compared to Trial 1, likely due to the use of multiple researchers to count shoots across Trial 2 treatments. Still, the variability between data points within Trial 2 is similar between the manual and automated treatments (R²=0.0354 and R²=0.1159; see, FIGS. 21 and 22) for comparison between the treatments. The results of Trial 2 (Table 7 column “Avg. Con shoot #/Total”) indicate similar variability between the automated and manual treatments, similar to Trial 1, but taking into account increased loss to contamination across Trial 2. The results of the sterilization method comparison between automated and manual methods indicate that automation of sterilization did not increase damage or death of prepared explants and that the viability of collected explants from automated sterilization were comparable to those in the manual method. Despite introduction of variables such as different researchers or batches of explants, the results support that the automated sterilization method does not reduce viability or alter explants when compared to manual sterilization.

### Example 2

In this example, excised explants were floated via the automated method described above and were compared to those floated via a conventional manual method, and viability of the explants was then compared. Viability of the explants floated via the automated method above were not adversely affected, as compared to viability of the explants floated via the conventional manual method.

In two separate trials, the two treatments (Automated Float and Manual Float) were initiated side-by-side. The Automated Float treatment was carried out as in Table 4 above. In the Manual Float treatment, about 6,840 explants were sterilized via swirling in 500 mL roller bottles containing 300 mL of sterilizing solution (70% ethanol containing 100 g/L PEG MW 8000 (polyethylene glycol)) for 3 minutes and 30 seconds. After, the sterilizing solution was drained and explants were manually rinsed with autoclaved water by pouring about 300 mL of water into the roller bottle, swirling the bottle to agitate the explants, and straining off the water into a sieve, and repeating for about 3 times. After rinse, the manual floatation method was performed by adding the explants and slowly adding 300 mL of sterile water into a 500 mL wide-mouthed beaker and allowing explants to float to the surface. The floating explants were poured off into a strainer, leaving debris behind. The manual float process was repeated until a visible excess of debris began to float, a visual assessment, usually 8-9 floats, and the remaining explant debris was discarded as waste. The Automated Float treatment was preceded by the manual sterilization and manual rinse methods as described for the Manual Float treatment in Example 1.

After completion of the float, explants were manually rehydrated and regenerated as described in Example 1. As in Example 1, after two weeks of regeneration, viable explants, those with the presence of a growing point at the meristematic region of the embryo, were counted.

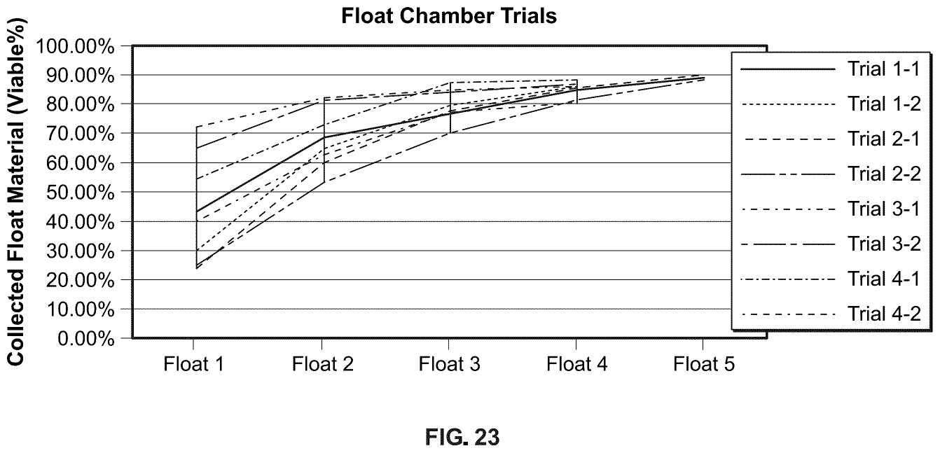

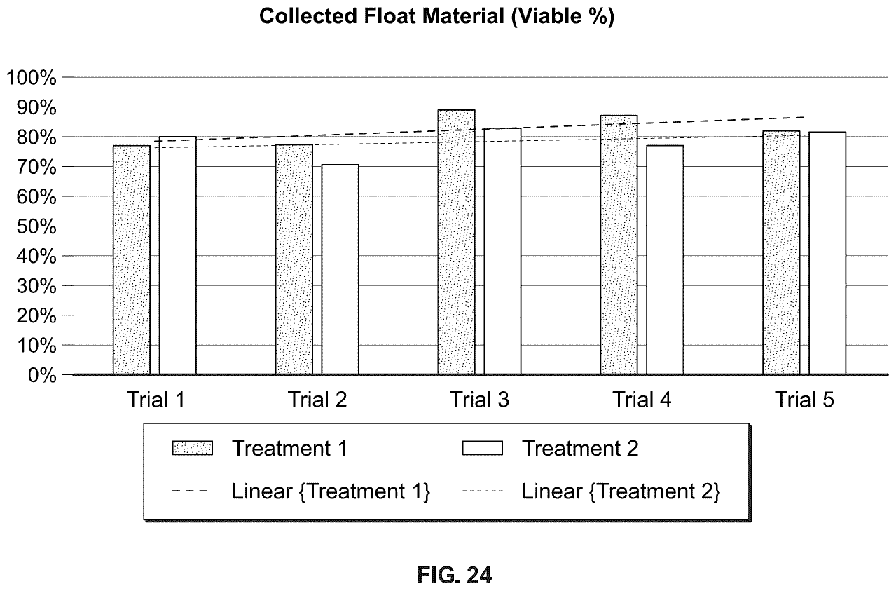

Five separate trials were initiated to compare the automated floatation method to the manual float method. Table 8 describes the trial setups, and FIGS. 23 and 24 represent the experimental results from the trials. Each consecutive trial is built on the information from the previous trial. The term “treatment” indicates a replicate of a trial in which a separate batch of explants was initiated in the experiment.

TABLE 8

| Trial | Treatment | Protocol Used |
|---|---|---|
| Trial 1 (about 6,840 | Automation | Two separate batches of explants |
| explants/treatment batch) | | (treatments) were put through 5 automated |
| | | float cycles for analysis. Treatment 1 |
| | | established a cycle for use and Treatment 2 |
| | | replicated that sequence to validate data. |
| Trial 2 (about 6,840 | Automation | Two separate batches of explants |
| explants/treatment batch) | | (treatments) were put through five automated |
| | | float cycles to validate cycle number analysis |
| | | from Trial 1. |
| Trial 3 (about 6,840 | Automation | Two separate batches of explants |
| explants/treatment batch) | | (treatments) were put through four |
| | | automated float cycles. |
| Trial 4 (about 75,000 | Automation | Two separate batches of explants |
| explants/treatment batch) | | (treatments) were put through four |
| | | automated float cycles. |
| Trial 5 | Manual | Manual floatation was performed as |
| | | described above using five repeated manual |
| | | floats. Two separate batches of explants |
| | | (treatments) were manually floated for |
| | | comparison. |

In connection with the above, FIG. 23 includes a comparison of each automated treatment (replicate) in each trial (1-4) as noted in Table 8. The x-axis represents the number of floatation cycles performed and the y-axis represents the Collected Float Material (Viable %). The Viable % is calculated as cumulative explants after float (g)/total collected explants (g). FIG. 24 includes a comparison of each of two treatments (batches) of the automated trials (Trials 1-4) with the manual floatation method (Trial 5). The y-axis represents the Collected Float Material (Viable %). The Viable % is calculated as cumulative explants after float (g)/total collected explants (g). The Viable % was calculated after three automated floats (Trials 1-4) or after the completion of all manual floats (Trial 5). Linear trendlines are shown for each treatment to compare trials.

That said, FIG. 23 illustrates that Trial 1 utilized five floatation cycles and a combination of rear and side jets for water agitation while Trial 2 utilized five floatation cycles and only the rear jet for water agitation. Results from these trials showed similar viability (y-axis) and little change in collected viable explants between float cycles 4 and 5, with values ranging from 88.06-90.46% viable explants collected. Trial 3 utilized four floatation cycles and the rear jet water agitation utilized in Trial 2. Trial 4 utilized four floatation cycles and the rear jet water agitation from Trial 2 on a large batch of explants (75,000). It was found that Trials 3 and 4 collected most of the viable explants (80.82-88.67%) in four floatation cycles. Throughout the course of these trials, a determination was made of the process (four automated float cycles and rear jet agitation) that was optimized for both large and small batch automated floatation collection of the viable explants.

FIG. 24 illustrates that all four experimental automated trials (Trials 1, 2, 3, and 4) had similar amounts of viable material collected via floatation as the standard manual method (Trial 5) across treatments. Treatment 1 of Trials 1-4 ranged from 77-89% collected viable float material over three floats while Trial 5 had 82% collected viable float material over all floats carried out. Treatment 2 of Trials 1-4 ranged from 71-83% collected viable float material over three floats while Trial 5 had 82% collected viable float material over all floats carried out. The dotted linear trendlines show low slopes (Treatment 1=0.0199; Treatment 2=0.0107), indicating similar viability capture across automated floatation and manual methods.

The results of the float viability testing for the automated method in FIGS. 23 and 24 indicate that the explants can be floated using the system 100 and that the explants collected from the automated floatation method were similar in viability to those of the manual method.

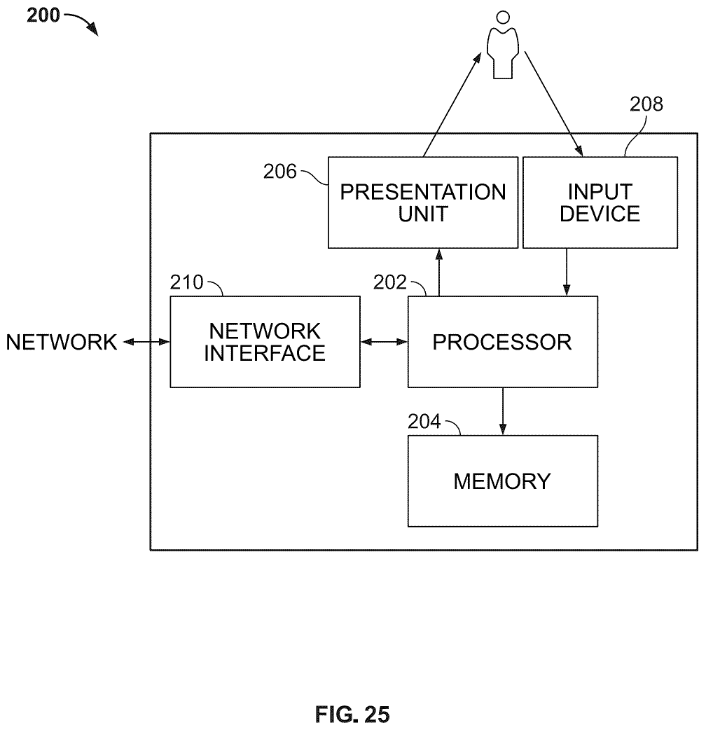

FIG. 25 illustrates an example computing device 200 that can be used in connection with the explant preparation system 100. The computing device 200 may include, for example, one or more servers, workstations, personal computers, laptops, tablets, smartphones, etc. In addition, the computing device 200 may include a single computing device, or it may include multiple computing devices located in close proximity or distributed over a geographic region, so long as the computing devices are specifically configured to function as described herein. In the example embodiment herein, the computing device 110 may be considered as including and/or being implemented in at least one computing device consistent with computing device 200. However, the present disclosure should not be considered to be limited to the computing device 200, as described below, as different computing devices and/or arrangements of computing devices and/or arrangement of components associated with such computing devices may be used.

Referring to FIG. 25, the example computing device 200 includes a processor 202 and a memory 204 coupled to (and in communication with) the processor 202. The processor 202 may include one or more processing units (e.g., in a multi-core configuration, etc.). For example, the processor 202 may include, without limitation, a central processing unit (CPU), a microcontroller, a reduced instruction set computer (RISC) processor, an application specific integrated circuit (ASIC), a programmable logic device (PLD), a gate array, and/or any other circuit or processor capable of the functions described herein.

The memory 204, as described herein, is one or more devices that permit data, instructions, etc., to be stored therein and retrieved therefrom. The memory 204 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable media. The memory 204 may be configured to store, without limitation, the various data (and/or corresponding data structures) described herein. Furthermore, in various embodiments, computer-executable instructions may be stored in the memory 204 for execution by the processor 202 to cause the processor 202 to perform one or more of the functions described herein, such that the memory 204 is a physical, tangible, and non-transitory computer readable storage media. Such instructions often improve the efficiencies and/or performance of the processor 202 and/or other computer system components configured to perform one or more of the various operations herein. It should be appreciated that the memory 204 may include a variety of different memories, each implemented in one or more of the functions or processes described herein.

The computing device 200 also includes a presentation unit 206 that is coupled to (and is in communication with) the processor 202 (however, it should be appreciated that the computing device 200 could include output devices other than the presentation unit 206, etc.). The presentation unit 206 outputs information to users of the computing device 200 as desired. And, various interfaces (e.g., as defined by network-based applications, etc.) may be displayed at computing device 200, and in particular at presentation unit 206, to display such information. The presentation unit 206 may include, without limitation, a liquid crystal display (LCD), a light-emitting diode (LED) display, an organic LED (OLED) display, an “electronic ink” display, speakers, etc. In some embodiments, the presentation unit 206 may include multiple devices.

In addition, the computing device 200 includes an input device 208 that receives inputs from the users of the computing device 200. The input device 208 may include a single input device or multiple input devices. The input device 208 is coupled to (and is in communication with) the processor 202 and may include, for example, one or more of a keyboard, a pointing device, a mouse, a touch sensitive panel (e.g., a touch pad or a touch screen, etc.), another computing device, and/or an audio input device. Further, in various exemplary embodiments, a touch screen, such as that included in a tablet, a smartphone, or similar device, may behave as both a presentation unit and an input device.

Further, the illustrated computing device 200 also includes a network interface 210 coupled to (and in communication with) the processor 202 and the memory 204. The network interface 210 may include, without limitation, a wired network adapter, a wireless network adapter, a mobile network adapter, or other device capable of communicating to one or more different networks. Further, in some example embodiments, the computing device 200 may include the processor 202 and one or more network interfaces incorporated into or with the processor 202.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “included with,” or “in communication with” another feature, it may be directly on, engaged, connected, coupled, associated, included, or in communication to or with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

None of the elements recited in the claims are intended to be a means-plus-function element within the meaning of 35 U.S.C. § 112 (f) unless an element is expressly recited using the phrase “means for,” or in the case of a method claim using the phrases “operation for” or “step for.”

The foregoing description of example embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An automated system for processing explant material, the automated system comprising:
a first station configured to sterilize explant material with sterilization media;
a second station configured to receive the explant material from the first station and rinse the explant material;
a third station configured to receive the explant material from the second station and hydrate the explant material with rehydration media; and
a support structure, wherein the first station, the second station, and the third station are positioned on the support structure.

2. The automated system of claim 1, wherein the second station is disposed generally between the first station and the third station.

3. The automated system of claim 1, wherein the first station is disposed generally higher than the second station, and wherein the second station is disposed generally higher than the third station.

4. The automated system of claim 1, wherein the first station, the second station, and the third station are included within a module, and wherein the module is removably coupled to the support structure.

5. The automated system of claim 1, wherein the first station includes a reservoir configured to hold the explant material and sterilization media; and
wherein the reservoir is configured to pivot relative to the support structure to transfer the explant material from the reservoir to the second station.

6. The automated system of claim 5, further comprising a paddle disposed within the reservoir, the paddle configured to move within the reservoir to mix the explant material and the sterilization media.

7. The automated system of claim 5, wherein the first station further includes a fluid discharge head positioned adjacent the reservoir, the fluid discharge head configured to deliver fluid to the reservoir.

8. The automated system of claim 1, wherein the second station includes a tank configured to receive the explant material from the first station and a fluid discharge head positioned adjacent the tank, the fluid discharge head configured to deliver fluid to the tank for use in rinsing the explant material received in the tank.

9. The automated system of claim 8, wherein the second station further includes at least one port positioned in a lower portion of the tank, the at least one port configured to deliver fluid to the tank to fill the tank with fluid and cause the explant material received in the tank to float on the fluid.

10. The automated system of claim 8, wherein the second station further includes at least one jet positioned in a wall of the tank, the at least one jet configured to deliver fluid to the tank to direct the explant material received in the tank to the third station.

11. The automated system of claim 1, wherein the third station includes a tank configured to receive the explant material from the second station.

12. The automated system of claim 11, wherein the third station further includes a storage chamber configured to hold the rehydration media and transfer the rehydration media to the tank for use in hydrating the explant material received in the tank of the third station.

13. The automated system of claim 1, further comprising a computing device configured to control one or more operations of the first station, the second station, and/or the third station.

14. The automated system of claim 1, wherein the second station is disposed generally between the first station and the third station;
wherein the first station is disposed generally higher than the second station, and wherein the second station is disposed generally higher than the third station.

15. The automated system of claim 14, wherein the first station, the second station, and the third station are included within a module, and wherein the module is removably coupled to the support structure.

16. An automated method for processing explant material, the automated method comprising:
sterilizing explant material within a reservoir of a first station of an automated explant processing system using a sterilization media;
actuating, by a computing device, the reservoir to transfer the sterilized explant material from the reservoir to a float tank of the system;
rinsing the explant material received within the float tank;
directing, via fluid flow within the float tank, the explant material from the float tank to a rehydration tank; and
hydrating the explant material received in the rehydration tank with a rehydration media.

17. The automated method of claim 16, wherein sterilizing the explant material within the reservoir includes rotating, by the computing device, a paddle within the reservoir to thereby mix the explant material with the sterilization media.

18. The automated method of claim 16, wherein actuating the reservoir includes pivoting the reservoir to thereby pour the explant material from the reservoir into the float tank.

19. The automated method of claim 16, wherein directing the explant material from the float tank to the rehydration tank includes:
directing, by at least one port disposed in a bottom portion of the float tank, fluid into the float tank to thereby cause the explant material to float on the fluid; and
directing, by at least one jet disposed in a sidewall of the float tank, fluid into the float tank to thereby direct the floating explant material out of the float tank and into the rehydration tank.

20. The automated method of claim 19, wherein sterilizing the explant material within the reservoir includes rotating, by the computing device, a paddle within the reservoir to thereby mix the explant material with the sterilization media; and
wherein actuating the reservoir includes pivoting the reservoir to thereby pour the explant material from the reservoir into the float tank.
